(19) United States
(12) Patent Application Publication
(10) Pub. No.: US 2025/0312027 A1
(43) Pub. Date: Oct. 9, 2025

(54) Injectable Soft Anchors and Methods of Tissue Repairs

(71) Applicant: Arthrex, Inc., Naples, FL (US)

(72) Inventors: Shane NOBLE, Naples, FL (US); Keith G. TAYLOR, Naples, FL (US); Thomas DOONEY, JR., Naples, FL (US); Chelsea DAY, Naples, FL (US); John M. TOKISH, Scottsdale, AZ (US)

(73) Assignee: Arthrex, Inc., Naples, FL (US)

(21) Appl. No.: 19/073,516

(22) Filed: Mar. 7, 2025

Related U.S. Application Data
(60) Provisional application No. 63/631,606, filed on Apr. 9, 2024.

(51) Int. Cl. A61B 17/04

(52) U.S. Cl. CPC A61B 17/0401; A61B 2017/0409; A61B 2017/0445; A61B 2017/0458; A61B 2017/0464

(57) ABSTRACT

Surgical fixation devices, constructs, tissue repairs and reconstructions, and methods of soft tissue fixation utilizing solidifying materials and biologics.

### BACKGROUND

The disclosure relates to the field of surgery and, more specifically, to surgical suture constructs and methods of tissue repairs for reconstructive surgeries.

### SUMMARY

Surgical constructs, fixation devices, and methods of tissue repairs are disclosed.

A suture anchor can be secured to bone with a solidifying material provided through vent holes alongside an inserter shaft and/or anchor sleeve. A solidifying material can include bonding material such as bone cement and/or biologics. A suture anchor can include an eyelet secured to an inserter shaft that injects biologics through its distal end. Flexible couplers from soft tissue are secured to the eyelet. The eyelet is inserted into a bone hole with the inserter. Solidifying material is injected through the inserter and flows through vent holes and into the bone. The solidifying material can fill the hole and provide fixation and integration of the flexible couplers to bone.

A suture anchor can be secured to bone with a solidifying material that is provided to a bone hole prior to anchor insertion.

Methods of tissue repairs are also disclosed. A knotless surgical construct can provide tissue to tissue fixation with increased fixation and with fewer passing steps.

### DESCRIPTION OF THE DRAWINGS

FIG. 1 illustrates a schematic cross-sectional view of a surgical assembly.

FIG. 2 illustrates an eyelet of the surgical assembly of FIG. 1.

FIG. 3 illustrates a front view of the surgical assembly of FIG. 1.

FIGS. 4-6 illustrate a tissue repair with the surgical assembly of FIG. 1.

FIG. 7 illustrates various flexible couplers.

FIGS. 8-10 illustrate another tissue repair.

FIGS. 11-13 illustrate schematic views of other surgical assemblies.

FIGS. 14-27 illustrate another tissue repair.

### DETAILED DESCRIPTION

The disclosure provides surgical fixation devices, constructs, assemblies, tissue repairs and reconstructions, and methods of soft tissue fixation utilizing solidifying materials and/or biologics.

Solidifying materials and/or biologics are utilized to fixate soft tissue to bone as a primary method of securing sutures which are passed through soft tissue to the intended insertion site. Solidifying materials and/or biologics provide augmentation of bone anchors for soft tissue fixation, as a minimally invasive method and/or biologically favorable option for improved osteointegration with respect to traditional bone suture anchors or all suture anchors generally.

In an embodiment, a device/apparatus can deliver biologics to the suture/tendon insertion site. The device can include an eyelet secured to a cannulated insertion sleeve that can inject biologics through the distal end. At the proximal end, the device has a feature to allow for suture to be tensioned to it, for the purpose of appropriately tensioning soft tissue fixated to suture. Further proximally, the device has a handle to allow for impaction and subsequent injection of biologics (solidifying material, bonding material) via a luer lock, after a stylet is removed. The stylet can then be used to force biologics through the injection ports. The eyelet and the solidifying materials and/or biologics act as a “suture anchor.”

In an embodiment, a suture anchor can be augmented with a solidifying material such as cement by cannulating the suture anchor inserter. The suture anchor can be a soft anchor or an “all-suture” anchor. The suture anchor can include an eyelet and an anchor body.

In an embodiment, a solidifying material such as cement can be used to augment a suture anchor rather than the solidifying material itself acting as the anchor.

As detailed below, the disclosure also provides different methods of using a solidifying material (such as bone cement) and/or biologics with suture anchors:
1) inject/provide the solidifying material/cement as the actual suture anchor;
2) inject/provide the solidifying material/cement through an anchor that is already implanted for additional fixation;
3) insert/provide solidifying material/cement through the suture anchor inserter during implantation of the anchor; and
4) inject/provide solidifying material/cement into a bone hole first, and then insert the anchor after.

Methods of tissue repairs with suture anchor constructs are also disclosed. An exemplary method includes inter alia the steps of: (i) securing at least one flexible strand to soft tissue to be attached to bone; (ii) attaching the at least one flexible strand to an eyelet secured to a cannulated inserter sheath; and (iii) injecting a solidifying material through vents in the cannulated sheath to secure the eyelet and the at least one flexible strand into a bone hole. The solidifying material acts as the actual suture anchor. The at least one flexible strand can be suture and the eyelet can be a suture eyelet.

Another exemplary method includes inter alia the steps of: (i) securing at least one flexible strand to soft tissue to be attached to bone; (ii) attaching the at least one flexible strand to an eyelet secured to an anchor; (iii) inserting the anchor and the eyelet with the attached at least one flexible strand into a bone hole; and (vi) injecting a solidifying material through the already implanted anchor. The at least one flexible strand can be suture and the eyelet can be a suture eyelet. The anchor can be any fixation device configured to engage and secure the eyelet and the at least one flexible strand into the bone hole. The anchor can be a cannulated screw with vent holes.

Another exemplary method includes inter alia the steps of: (i) securing at least one flexible strand to an anchor body attached to an inserter; (ii) inserting the anchor body with the at least one flexible strand into a bone hole; and (iii) injecting a solidifying material through the inserter during anchor implantation/insertion. The anchor body can be a soft body anchor (such as an “all-suture” anchor) or a hard body anchor. The at least one flexible strand can be suture attached to soft tissue to be attached to bone.

Another exemplary method includes inter alia the steps of: (i) injecting a solidifying material into a bone hole; and (ii) subsequently, inserting a fixation device with at least one flexible strand attached to soft tissue into the bone hole. The at least one flexible strand can be suture. The at least one flexible strand can be previously secured/attached to another fixation. The another fixation device can be an “all-suture” soft anchor inserted into another bone hole.

The one or more tissue repair applications can include soft tissue repairs; orthopedic surgical repairs such as rotator cuff repairs, Achilles tendon repairs, patellar tendon repairs, ACL/PCL reconstructions, hip and shoulder reconstructions, among many others.

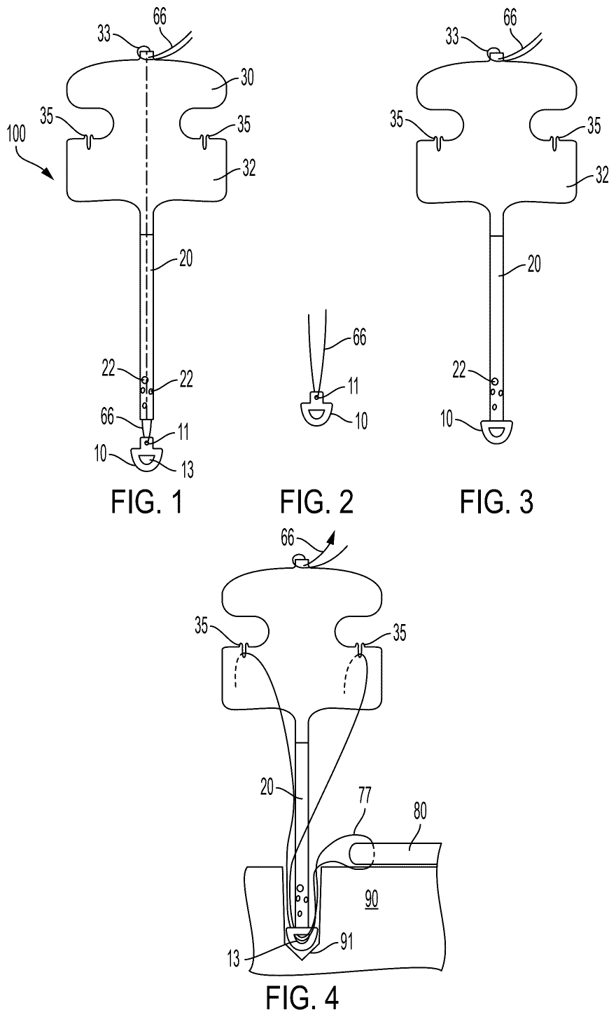

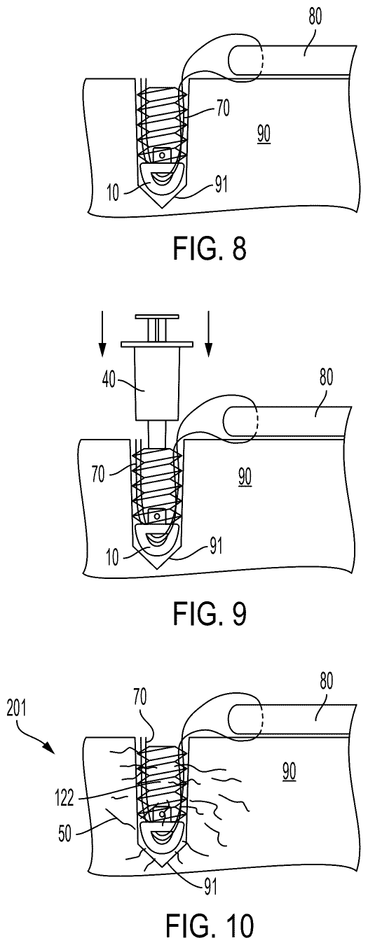

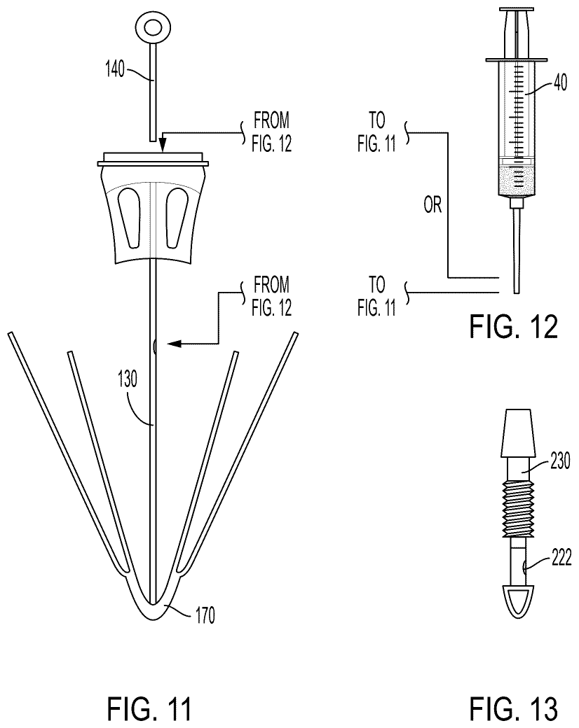

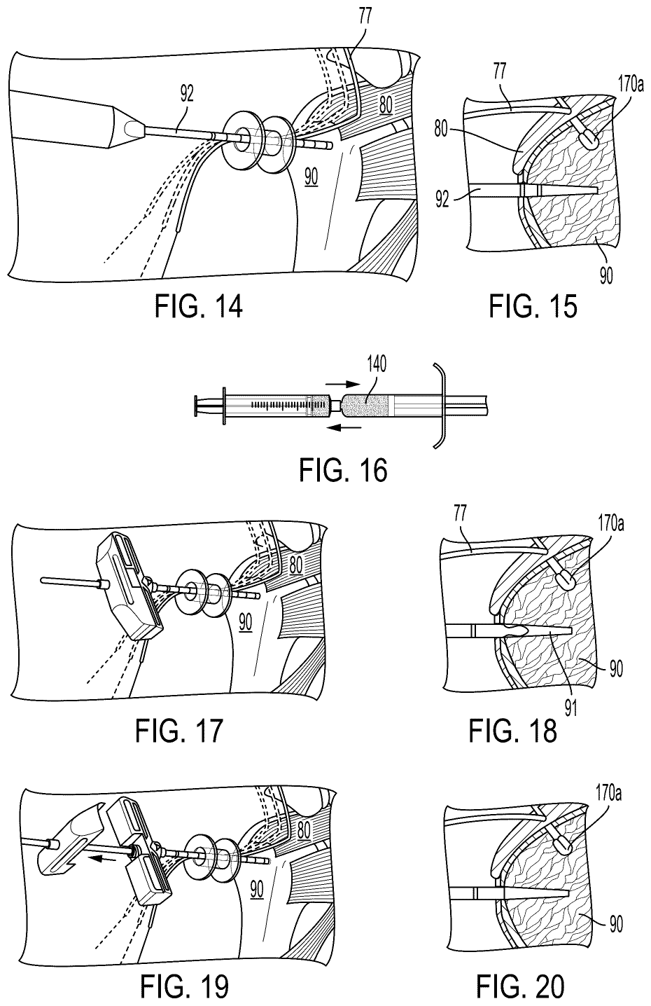

Referring now to the drawings, where like elements are designated by like reference numerals, FIGS. 1-27 illustrate exemplary fixation devices, assemblies, and surgical repairs of the present disclosure. FIGS. 1-7 illustrate schematic steps of tissue repairs 101 with device 100. FIGS. 8-10 illustrate schematic steps of tissue repairs 201. FIGS. 11-13 illustrate views of fixation devices used with solidifying materials and/or biologics. FIGS. 14-27 illustrate schematic steps of tissue repairs 301.

The embodiments detailed below will be described with reference to a solidifying material 50 (bonding material 50; bondable material 50; binding material 50; biomaterial 50; fillable material 50; filler 50; cement 50; adhesive 50; binding biologics material 50). The solidifying material 50 can be absorbable or non-absorbable. The solidifying material 50 can be a polymer, liquid polylactic acid (PLA), polyetheretherketone (PEEK), metal, liquified biomaterial, or any other liquid or flowable material or combinations thereof. Solidifying material 50 can include a bone cement or any bone substitute materials that can cement and/or augment hard tissue during filling of hard tissue defects such as bone holes, for example. Solidifying material 50 can also provide a mechanism for hard tissue (bone) ingrowth in the form of an injectable biocompatible material (composite). Solidifying material 50 can be any material or combination of materials that can replace bone and/or hard tissue. Solidifying material 50 can be any substitute material that has physical and biological properties about similar to those of the patient's own bone.

Solidifying material 50 can comprise bone cement which can include liquid or fluid mixtures consisting of hydroxyapatite (HA), tri- and tetra-calcium phosphate, harvested bone autografts and/or allografts, and/or an acidic, aqueous liquid carrier that can be applied to the bone or into a bone hole or defect site. The solidifying material 50 can harden after few seconds or over a period of time, filling the bone hole and cementing the structures (sutures, flexible strands, fixation devices, etc.) implanted into the bone hole.

Solidifying material 50 can comprise one or more composite materials in the form of particles, granules, and/or powder of calcium-containing compound such as calcium phosphate bone void fillers. Solidifying material 50 can include a matrix of a biocompatible materials that can be employed as a bone substitute or a bone cement and that can optionally act as a scaffold for bone ingrowth.

Solidifying material 50 can include an injectable composition such as Arthrex Quickset™ calcium phosphate cement (Quickset™ bone cement) which is a macroporous, injectable, hardening and resorbable bone filler provided in an easy-to-use, closed mixing system. The mixing system is a dual-chambered syringe containing a powder and mixing liquid. The powder chamber contains a mixture of calcium phosphates and an organic polysaccharide polymer; the polysaccharide is a highly biocompatible polymer that optimizes the viscosity, cohesiveness, and macroporosity. The mixing liquid consists essentially of a sodium phosphate solution that facilitates the setting time (crystallization) of the cement.

Solidifying material 50 can include an injectable composition such as Arthrex BoneSync™ cement (a calcium phosphate cement) designed to fill bony voids or gaps in bones and which can be cured in situ and is fast-setting. The BoneSync™ cement can be mixed with saline, blood, and/or bone marrow aspirate, to form an easy-to-use, fast remodeling, settable, and drillable biomimetic solution to tissue repair. The BoneSync™ cement can also include a collagen additive to improve integration with native bone and maintain volume while setting. The BoneSync™ cement can be also hydrated with biologic fluids, such as BMA (bone marrow aspirate). The BoneSync™ cement is a fast-setting, synthetic bone void filler that sets within about 5 to about 8 minutes, depending on whether it is prepared with saline, blood, or bone marrow.

Solidifying material 50 can consist essentially of one or more liquified materials (liquified biomaterials). In certain embodiments, solidifying material 50 can consist of one or more liquified materials (liquified biomaterials).

Solidifying material 50 can include biologics in addition to, or in lieu of, the bone cement/bone substitute. Solidifying material 50 can include ACP (autologous conditioned plasma), PRP (platelet rich plasma), and/or BMA (bone marrow aspirate), which provide a cell suspension that can be readily processed intraoperatively for immediate implantation. BMA can be also injected directly to a repair site (bone hole), before or during cement injection. Additional biologics can be also injected directly to a repair site, before or during cement injection.

FIG. 1 illustrates device 100 with eyelet 10 secured to cannulated shaft 20 of inserter 30 by flexible coupler 66. Inserter 30 includes a handle 32 connected to cannulated inserter shaft 20. A plurality of vent holes 22 (vents 22; openings 22; apertures 22; holes 22; gaps 22) are provided alongside of inserter shaft 20 and at a distal end 23 of the inserter shaft 20. Handle 32 is also provided with slots 35 to allow suture attached to soft tissue to be secured therein. Vent holes 22 are provided for the flow of solidifying material and/or biologics 50, as detailed below.

Eyelet 10 is configured with two openings 11, 13 designed to allow flexible couplers 66, 77, respectively, to slidably pass through and as detailed below. Eyelet 10 includes a proximal end 12 that houses opening 11 and a body 15 that houses opening 13. Proximal end 12 is configured to be received within distal end 23 of inserter shaft 20 upon advancement of the inserter shaft 20 over proximal end 12 of the eyelet 10 (FIG. 3). Eyelet 10 can be press fit or threaded on distal end 23 of inserter shaft 20.

Flexible coupler 66 (retention stitch 66; retention suture 66; first flexible coupler 66) is slidably attached to eyelet 10 by passing through opening 11, as shown in FIG. 2. To secure eyelet 10 to inserter 30, flexible coupler 66 can be passed through the cannulated inserter shaft 20 and cannulated handle 32 and attached to a securing device 33 on handle 32. FIG. 3 illustrates device 100 in a fully assembled position, i.e., an assembled anchor driver position.

Reference is now made to FIG. 4 which illustrates device 100 employed in a soft tissue repair 101 (FIG. 6). Once eyelet 10 has been secured to inserter 30, at least one flexible coupler 77 attached to soft tissue 80 is passed through opening 13 of eyelet 10 for anchoring into bone 90. FIG. 4 illustrates eyelet 10 with flexible coupler 77 and most distal end 23 with vents 22 inserted into bone hole 91 (bone opening 91). Flexible coupler 77 (tissue suture 77; soft tissue suture 77; second flexible coupler 77; suture 77; flexible strand 77) can include sutures from tissue 80 which are inserted into the eyelet 10. The eyelet 10 is inserted into prepared bone hole 91 and the suture tension is finalized. The sutures 77 can be cleated to the handle 32 (on handle slots 35, for example) to maintain the tension. Retention suture 66 is removed once suture tension is finalized.

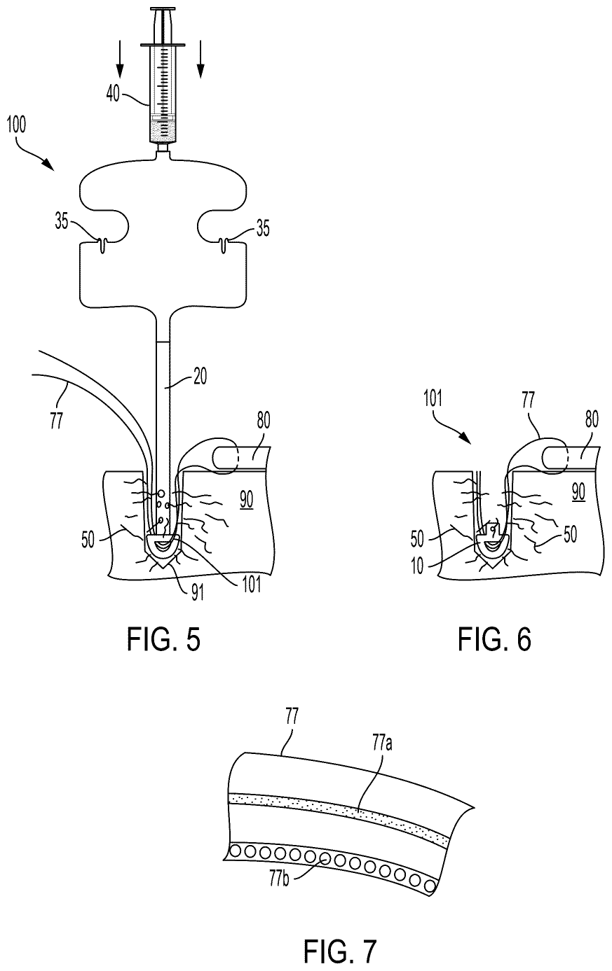

FIG. 5 illustrates the insertion/injection of solidifying material 50 through the handle and driver with exemplary device 40 (syringe 40; injection device 40; needle 40). As detailed above, solidifying material 50 can comprise cement (bone cement or fixation cement) and/or biologics. The cement 50 flows through the vent holes 22 and into the bone hole 91. In the exemplary embodiment of FIGS. 1-6, the cement 50 is the actual suture anchor. The sutures 77 are removed from the cleats 35 and the handle/driver is backed up. During removal, injecting can continue to back fill the hole 91. The sutures 77 can be cut to complete repair 101 (FIG. 6). Alternatively, sutures 77 can be employed in additional steps, for example, to be secured with additional fixation devices and/or to be employed for any additional surgical repairs.

In the repair 101 of FIG. 6, the solidifying material 50 acts as an actual suture anchor to anchor sutures 77 to bone 90. Cement 50 can back fill the bone hole 91 and once “set” provides the necessary fixation and integration of the sutures 77 to bone 90. In repair 101 of FIG. 6, eyelet 10 simply positions the suture 77 to the bottom of the hole 91 so that the suture can be tensioned; the eyelet does not provide fixation in this embodiment.

FIG. 7 illustrates various configurations of flexible coupler 77 that can be employed with the disclosure. Round suture 77, flat suture 77a, and/or “porous woven” suture 77b can be used for this application. Porous woven sutures 77b can provide the optimal structure for the solidifying material 50 (cement 50) to integrate through its vent holes.

An exemplary round suture 77 can be FiberWire® suture, sold by Arthrex, Inc. of Naples, Fla., and described in U.S. Pat. No. 6,716,234, the disclosure of which is incorporated by reference herein. An exemplary flat suture 77a can be a tape such as FiberTape® suture tape, as disclosed in U.S. Pat. No. 7,892,256, the disclosure of which is incorporated in its entirety herewith. An exemplary porous woven suture 77b can be any of the sutures described and illustrated in U.S. application Ser. No. 17/996,509 filed Mar. 21, 2021, to Linden et al. and assigned to Arthrex, Inc., the disclosure of which is incorporated by reference in its entirety herewith.

Suture 77b can be in the form of suture tape with a plurality of vent holes 22a (vents 22a; openings 22a; through holes 22a; perforations 22a; holes 22a). Holes 22a confer increased porosity to suture 77b for achieving a better integration of the suture with soft tissue and with the solidifying material (cement), and for also increasing the strength of the overall repair. In an embodiment, vent holes 22a can be provided in a regular pattern along the body of the suture/tape. In an embodiment, vent holes 22a can be provided irregularly along the body of the suture/tape. Vent holes 22a can have similar or different dimensions, and can have similar or different configurations. Suture 77b can also include a combination of suture region(s) with vents 22a having similar dimensions and suture region(s) with vents 22a having different dimensions. Vent holes 22 increase the porosity of suture 77b and allow the solidifying material 50 to easily pass through (and through the suture 77b) to allow enhanced fixation. In an embodiment, suture 77b can include a material with a porosity greater than about 25% (i.e., more than about 25% porous). In an embodiment, suture 77b can include a material with a porosity greater than about 50% (i.e., more than about 50% porous).

FIGS. 8-10 illustrate steps of tissue repair 201 (FIG. 10) wherein solidifying material 50 is injected through a fixation device 70 that has been already implanted/inserted into bone hole 91. In an exemplary-only embodiment, cannulated screw 70 (anchor 70) secures eyelet 10 with attached flexible couplers 77 within bone hole 91. As detailed above, flexible coupler(s) attach soft tissue 80 to bone 90. Solidifying material 50 (cement 50) is injected with device 40 (FIG. 9) through the already-implanted anchor 70, for additional fixation. Cannulated screw 70 (anchor 70) can be provided with vent holes 122 (similar to vent holes 22 detailed above with reference to inserter shaft 20) to allow flow of cement 50 within bone hole 91, around and within eyelet 10 (through opening 13 which allows passage of flexible coupler 77), within the lumen of cannulated anchor 70, and around flexible coupler 77 extending along outer walls of the cannulated anchor 70.

FIGS. 11-13 illustrate additional embodiments wherein the solidifying material 50 is inserted through suture anchor inserter 130 during implantation of anchor 170 (fixation device 170). As shown in FIG. 11, suture anchor inserter 130 is a cannulated inserter provided with canulated side port(s) 122 for escape of injection of solidifying material 50 (cement 50). Plunger 140 (FIG. 11) and/or injection device 40 (syringe 40) (FIG. 12) can be employed to insert the cement through the anchor inserter 130 to secure anchor 170. In an exemplary-only embodiment, fixation device 170 is a soft anchor such as an “all-suture” anchor. A soft anchor (soft suture anchor or all-suture soft knotless anchor) is provided with a soft anchor sleeve (sheath or tubular member) with two open ends, and at least one flexible strand extending through the soft anchor sleeve (sheath). The flexible strand(s) may extend through the sleeve in similar or different directions and/or orientations and/or locations. The flexible strands can be suture, tapes such as suture tape, suture chains and/or any flexible couplers that allow attachment of soft tissue to bone. The flexible tubular sleeve with the flexible strands may be secured into or onto bone, the flexible strands may pass over or around soft tissue (such as rotator cuff) and can be secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

FIG. 13 illustrates an additional embodiment of an Arthrex SwiveLock® anchor inserter 230 provided with one or more holes 222 (vent holes 222) in the inserter to secure a fixation device such as a SwiveLock® anchor, disclosed in U.S. Pat. Nos. 8,012,174 and 9,005,246, the disclosures of both of which are fully incorporated by reference in their entirety herein. The fixation devices may be any of swivel and/or screw-in suture anchors and/or push-in suture anchors (such as s PushLock® anchor, as disclosed in U.S. Pat. No. 7,329,272). The fixation devices may be also any anchors, implants or screws (such as interference screws or tenodesis screws) or any fixation element that allows attachment/fixation of the soft tissue to bone.

Reference is now made to FIGS. 14-17 which illustrate another exemplary method of tissue repair 301 (FIGS. 26 and 27). An exemplary sequence of method steps is set forth below:

### FIGS. 14-27 Anchor Augmentation With BoneSync™ Calcium Phosphate Cement

### FIGS. 14 and 15:

Prepare a bone socket 91 with a punch 92 such as, for example, a SwiveLock® punch. FIG. 15 illustrates exemplary fixation device 170a in the form of a soft suture anchor 170a similar to soft anchor 170 detailed above. Flexible strands 77 are illustrated passed through soft tissue 80 to be attached to bone 90.

### FIG. 16:

Draw about 0.8 mL of saline into an empty syringe 140. Attach BoneSync™ syringe 140 with loosen powder to the empty syringe and transfer the material back and forth allowing the fluid to mix fully with the powder for about 60 seconds. When fully mixed, transfer the BoneSync™ mixture 50 into the original saline syringe. BoneSync™ cement 50 can be injected after mixing.

### FIGS. 17-20:

Insert a delivery needle into the hole 91, slightly past the cortex and remove stylet.

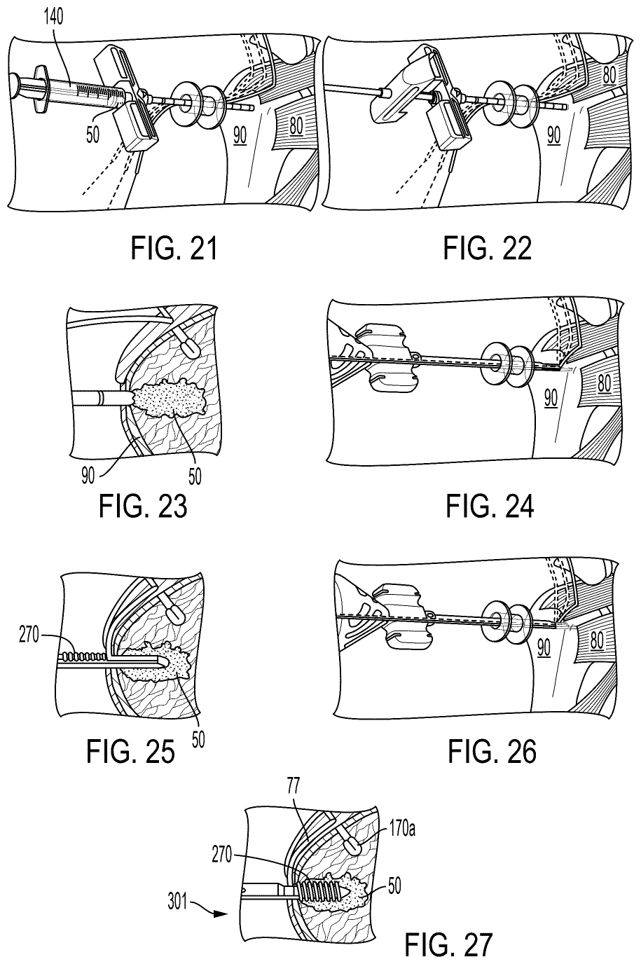

### FIGS. 21-23:

Insert the syringe 140 to luer fitting of the delivery needle and inject BoneSync™ cement 50. A trocar can be inserted into delivery needle to deliver the remaining material 50.

### FIGS. 24-27:

A fixation device 270 is attached to suture 77 and inserted into desired location and into cement 50 for repair 301. Steps can be repeated as necessary. Fixation device 270 can be an anchor (knotted anchor, knotless anchor, or all-suture anchor), implant, button, screw or any fixation device that confers secure attachment and fixation of soft tissue 80 to bone 90. The fixation device 270 can be a knotless suture anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein. Another exemplary fixation device 270 is a soft anchor or an “all-suture” anchor.

A suture anchor can be secured to bone with a solidifying material provided through vent holes alongside an inserter shaft and/or anchor sleeve. A solidifying material can include bone cement and/or biologics and/or similar materials. A suture anchor can include an eyelet secured to a cannulated insertion sleeve that injects biologics through its distal end. Flexible couplers from tissue are secured to the eyelet and the eyelet is inserted into a bone hole with an inserter. Solidifying material is injected through a handle of the inserter and through the inserter, and flows through vent holes and into the bone. The solidifying material can fill the hole and once is set can provide fixation and integration of the flexible couplers to bone.

A suture anchor can be secured into a bone hole with a solidifying material that is provided to the bone hole prior to anchor insertion. A solidifying material can be bone cement that is inserted into a prepared bone hole and then an anchor is inserted into the bone cement.

Cannulated anchor inserters with screw on/or side ports for solidifying material (such as calcium phosphate) insertion are also provided. In lieu of, or in addition to, a cannulated needle to introduce calcium phosphate into the medial row of a FiberTak® anchor, the anchor inserter is cannulated. In this manner, all suture anchors can deliver calcium phosphate into anchor tunnel at once (i.e., no need to remove the anchor to put in a needle-inject it through the inserter). Medial row FiberTak® anchor can be inserted and then calcium phosphate can be placed in any and/or all anchors. A cannulated anchor inserter with screw on/or side ports can be employed for any fixation devices such as SwiveLock® anchors and/or glenoid remplissage anchors that occasionally pull out. The inserter can be cannulated to allow the injection of solidifying material into the bone that exits just above and leaks somewhat down around the anchor, giving all anchors with questionable fixation an option for calcium phosphate augmentation, with insertion right through the anchor's cannulated inserter. The inserter is provided with an injection port for a simple solution to delivery of calcium phosphate. The injection port can be located on top, or it can be located lower on the inserter with a side port (hole) to insert the needle or attach (for example, by screwing in) a syringe into the inserter.

The methods and devices described above can be used in various soft tissue repairs or fixations and for attaching tissue (e.g., ligament, tendon, graft, etc.) to bone. For example, the devices and materials described above can be used in conjunction with a variety of orthopedic surgical repairs, including but not limited to rotator cuff repairs, Achilles tendon repairs, patellar tendon repairs, ACL/PCL reconstructions, hip and shoulder reconstructions, among many others.

Flexible coupler 77, 77a, 77b of repairs 101, 201, 301 can be employed for attachment of soft tissue 80 to bone 90 by passing the flexible coupler through or around soft tissue and forming at least one adjustable, flexible, continuous, tensionable loop around or through soft tissue 80. The at least one adjustable, flexible, continuous, tensionable loop around or through soft tissue 80 can be knotted or knotless. One or more flexible couplers 77, 77a, 77b and optional shuttling strands may extend through the lumen of the fixation devices to be implanted and secured with solidifying, bonding material 50 into bone 90.

Flexible coupler 77, 77a, 77b can be further attached to one or more fixation devices such as any anchors, for example, knotted anchors, knotless anchors, or all-suture anchors, or any devices that confer secure attachment and fixation of soft tissue 80 over bone 90. As detailed above, the fixation device can be a knotless anchor such as a two-piece Arthrex PushLock® anchor, or an Arthrex SwiveLock® anchor, or another all-suture soft anchor. Flexible coupler 77, 77a, 77b can consist essentially of suture. Flexible coupler 77, 77a, 77b can consist essentially of elastic suture.

An exemplary method of soft tissue reconstruction 101 comprises inter alia the steps of: (i) securing at least one flexible strand 77, 77a, 77b to soft tissue 80 to be attached to bone 90; (ii) attaching the at least one flexible strand 77, 77a, 77b to an eyelet 10 secured to a cannulated inserter shaft 20; and (iii) providing a solidifying material 50 through vents 22 in the cannulated inserter shaft 20 to secure the eyelet 10 and the at least one flexible strand 77, 77a, 77b into a bone hole 91. The step of providing a solidifying material 50 through vents 22 can include injecting the solidifying material 50 through vents 22. The solidifying material 50 can act as the actual suture anchor. The at least one flexible strand 77, 77a, 77b can be suture and the eyelet 10 can be a suture eyelet 10. The at least one flexible strand 77, 77a, 77b can be round suture 77, flat suture 77a, porous suture 77b, or combinations thereof.

Another exemplary method of soft tissue reconstruction 201 comprises inter alia the steps of: (i) securing at least one flexible strand 77, 77a, 77b to soft tissue 80 to be attached to bone 90; (ii) attaching the at least one flexible strand 77, 77a, 77b to an eyelet 10 secured to an anchor 70; (iii) inserting the anchor 70 and the eyelet 10 with the attached at least one flexible strand 77, 77a, 77b into a bone hole 91; and (vi) providing a solidifying material 50 through (and to) the already implanted anchor 70, 10. The step of providing a solidifying material 50 can include injecting the solidifying material 50 through vents 122 provided within anchor 70. The at least one flexible strand 77, 77a, 77b can be suture and the eyelet 10 can be a suture eyelet.

Another exemplary method of soft tissue reconstruction comprises inter alia the steps of: (i) securing at least one flexible strand to an anchor body 170 attached to an inserter 130; (ii) inserting the anchor body 170 with the at least one flexible strand into a bone hole 91; and (iii) injecting a solidifying material 50 through the inserter 130 during anchor implantation/insertion. The anchor body 170 can be a soft body anchor 170 (such as an “all-suture” anchor 170). The at least one flexible strand can be suture attached to soft tissue 80 to be attached to bone 90.

Another exemplary method of soft tissue reconstruction 301 comprises includes inter alia the steps of: (i) providing a solidifying material 50 into a bone hole 91; and (ii) subsequently, inserting a fixation device 270 with at least one flexible strand 77, 77a, 77b attached to soft tissue 80 into the bone hole 91. The step of providing a solidifying material 50 can be conducted by injecting the solidifying material 50. The at least one flexible strand 77, 77a, 77b can be suture. The at least one flexible strand 77, 77a, 77b can be further attached to a fixation device 170a and secured in bone 90 at a separate location. Device 170a can be a soft anchor such as an “all-suture” anchor.

Tissue repairs 101, 201, 301 can be knotted or knotless repairs. Flexible coupler 77 can form at least one adjustable, flexible, knotless, continuous, tensionable loop around soft tissue 80. Flexible coupler 77, 77a, 77b can be secured to knotted and/or knotless fixation devices. The fixation device can be a hard body anchor. The fixation device can be a soft anchor. The at least one flexible coupler 77, 77a, 77b can consist essentially of elastic suture. The soft tissue 80 can be rotator cuff. A plurality of soft fixation devices can be used with same or different solidifying materials 50 for a soft tissue repair, such as a rotator cuff repair.

Flexible coupler 66, 77, 77a, 77b can be a suture. Non-limiting examples of suitable sutures include FiberWire®, TigerWire®, or FiberChain® suture, although any type of suture may be utilized, including cored or coreless sutures. In another embodiment, flexible coupler 66, 77, 77a, 77b can be suture tape, such as FiberTape®. Flexible coupler 66, 77, 77a, 77b can include any soft, flexible strand of material.

Flexible coupler 66, 77, 77a, 77b can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided optionally with at least one other elastic fiber, natural or synthetic, to form lengths of suture material. Flexible coupler 66, 77, 77a, 77b can also include suture tape such as FiberTape® suture tape (as disclosed in U.S. Pat. No. 7,892,256, the disclosure of which is incorporated in its entirety herewith) or collagen tape, or wide “tape like” material, or combinations thereof.

Flexible coupler 66, 77, 77a, 77b can consist essentially of suture material and elastane, or combination of suture material and elastane and other materials such as long chain synthetic polymers like polyester and nylon, or materials such as PET, silk nylon or absorbable polymers, or coating materials (such as wax, silk, or silicone products), among many others. Flexible coupler 66, 77, 77a, 77b can consist of strands with cross-sections of various forms and geometries, including round, oval, rectangular, or flat, among others, or combinations of such forms and geometries.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A method of soft tissue repair comprising:
securing at least one flexible coupler to a fixation device;
securing the fixation device to an inserter shaft, wherein the inserter shaft is cannulated and provided with a plurality of vent holes;
inserting the fixation device into a bone hole with the inserter shaft; and
providing a solidifying material through the plurality of vent holes of the inserter shaft, to secure the at least one flexible coupler within the bone hole.

2. The method of claim 1, wherein the at least one flexible coupler is attached to soft tissue to be secured to bone.

3. The method of claim 1, wherein the at least one flexible coupler is suture, suture tape, or porous suture.

4. The method of claim 1, wherein the solidifying material includes at least one bonding material.

5. The method of claim 1, wherein the solidifying material includes bone cement.

6. The method of claim 1, wherein the fixation device is an eyelet with a through-opening, and wherein the method further includes slidingly passing the at least one flexible strand through the through-opening of the eyelet.

7. The method of claim 1, wherein inserting the fixation device into a bone hole is conducted with the inserter shaft.

8. The method of claim 1, wherein the soft tissue is rotator cuff.

9. The method of claim 1, wherein the soft tissue repair is a knotless repair.

10. A method of soft tissue repair comprising:
securing at least one flexible coupler to soft tissue to be attached to bone;
securing the at least one flexible coupler to a fixation device, wherein the fixation device has a cannulated anchor body provided with a plurality of vent holes;
securing the fixation device with the at least one flexible coupler into a bone hole; and
subsequently, providing a solidifying material through the plurality of vent holes of the fixation device, to secure the at least one flexible coupler within the bone hole.

11. The method of claim 10, wherein the solidifying material consists essentially of one or more liquified materials.

12. The method of claim 10, further comprising passing the at least one flexible strand through a suture eyelet and attaching the suture eyelet to the fixation device.

13. The method of claim 10, wherein providing a solidifying material is conducted by injecting the solidifying material with an injection device and through a plurality of through-holes in the at least one flexible coupler.

14. A method of soft tissue repair comprising:
securing at least one flexible coupler to soft tissue to be attached to bone;
providing a solidifying material into a bone hole formed into the bone;
subsequently, inserting the at least one flexible coupler into the bone hole and into the solidifying material, to secure the at least one flexible coupler within the bone hole.

15. The method of claim 14, further comprising:
attaching the at least one flexible coupler to a first fixation device;
securing the first fixation device into bone;
passing the at least one flexible coupler through or around the soft tissue;
securing the at least one flexible coupler to a second fixation device; and
securing the second fixation device with the at least one flexible coupler into the bone hole and into the solidifying material.

16. The method of claim 15, wherein the first fixation device is an all-suture anchor.

17. The method of claim 15, wherein the second fixation device is a hard-body anchor.

18. The method of claim 14, wherein the solidifying material is bone cement.

19. The method of claim 14, wherein the soft tissue repair is rotator cuff repair.

20. The method of claim 14, wherein providing a solidifying material is conducted by injecting the solidifying material with an injection device.

21. A soft tissue repair comprising:
a knotless soft suture anchor secured at a first location within bone;
a fixation device secured at a second location within the bone; and
a flexible coupler extending between the knotless soft suture anchor and the fixation device, wherein the flexible coupler passes through soft tissue to be attached to the bone, and wherein the second location is a bone hole with a solidifying material.

22. The soft tissue repair of claim 21, wherein the solidifying material is liquified biomaterial and wherein the fixation device is one of a push-in anchor, press-in anchor, or swivel anchor.

23. The soft tissue repair of claim 21, wherein the flexible coupler is a porous suture provided with a plurality of perforations or through-holes to allow the solidifying material to pass through.
